(19) United States
(12) Patent Application Publication
(10) Pub. No.: US 2021/0407080 A1
(43) Pub. Date: Dec. 30, 2021

(54) Systems and Methods for Characterizing Cells and Microenvironments

(71) Applicant: Arrive PTE Ltd., Singapore (SG)

(72) Inventors: Evan Szu, Belmont, CA (US); Nishant Borude, San Francisco, CA (US); Nivedita Suresh, San Francisco, CA (US); Michael H. Chu, Austin, TX (US); David G. Zapol, San Francisco, CA (US); Vinona Bhatia, San Francisco, CA (US); Darick M. Tong, San Francisco, CA (US); Noriko Y. Tong, San Francisco, CA (US); John Cheng, El Cerrito, CA (US); Clifford Szu, Zephyr Cove, NV (US); Eric J. Suba, San Francisco, CA (US)

(21) Appl. No.: 17/357,411

(22) Filed: Jun. 24, 2021

Related U.S. Application Data
(60) Provisional application No. 63/044,148, filed on Jun. 25, 2020.
(60) Provisional application No. 63/050,342, filed on Jul. 10, 2020.

(51) Int. Cl. G06T 7/00; G16H 50/20

(52) U.S. Cl. CPC G06T 7/0012; G06T 2207/30024; G16H 50/20

(57) ABSTRACT

Cell identification and classification is a well-known problem in the pathology domain that help identify microenvironments. In addition to the characteristic of each cell, its interactions with the neighboring regions or other cells is also important. This involves correct identification of neighboring elements and analytically representing the interactions between them. This disclosure presents a system that combines many such features, some hand engineered and some machine derived through training of Deep Learning algorithms that can be used to study the microenvironments.

### CROSS-REFERENCE TO RELATED APPLICATIONS

This application claims priority to and the benefit of U.S. Provisional Patent Application No. 63/044,148, filed Jun. 25, 2020 and U.S. Provisional Patent Application No. 63/050,342, filed Jul. 10, 2020, each of which are incorporated herein by reference in their entireties.

### FIELD OF TECHNOLOGY

The presently disclosed embodiments relate to systems and methods for characterizing cells, microenvironments, and structures, and more particularly to feature derivation and selection for in silico assays leveraging tumor, immune cell, microenvironment, fibrosis, necrosis, scarring, and/or structural characterization.

### SUMMARY OF DISCLOSURE

The present disclosure includes systems and methods to predict clinical and experimental outcomes by characterizing immune cells and their associated microenvironments and structures through a novel integration of hand-engineered and machine-determined complex features. Through machine learning synthesis of independently derived histologic, geolocational, and sequencing features, a more nuanced and complete view of immune infiltration into tissues is operationalized than existing methods. Such an approach enables unique inference extrapolation to non-identical settings and utilization of algorithmic outputs as intermediate outcome proxies for clinical/experimental outcomes in novel settings.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIGS. 1-9 show one or more schematic flow diagrams, certain computer-based architectures, and/or screenshots of various specialized graphical user interfaces which are illustrative of some exemplary aspects of at least some embodiments of the present disclosure.

FIG. 1 shows a flowchart of an illustrative methodology for training machine learning models for automatic feature detection for characterization of a microenvironment;

FIG. 2 shows a flowchart of an illustrative methodology for implementing machine learning models for automatic feature detection for characterization of a microenvironment;

FIG. 3 shows a flowchart of an illustrative methodology for implementing machine learning models for determining correlations between detected features for characterization of a microenvironment and patient outcomes;

FIG. 4 shows an illustration of tissue infiltration of target cells in three-dimensional stacked images as determined by the machine learning model approach described in FIGS. 1-3;

FIG. 5 shows an illustration of more efficient extrapolation of distance of cells and structural volumes from tissue sections cut across divergent axes using the machine learning model approach described in FIGS. 1-3;

FIG. 6 shows a schematic of an exemplary computer-based system and platform;

FIG. 7 shows a block diagram of another exemplary computer-based system and platform;

FIG. 8 shows a schematic of exemplary implementations of the cloud computing/architecture(s); and

FIG. 9 shows another schematic of exemplary implementations of the cloud computing/architecture(s).

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

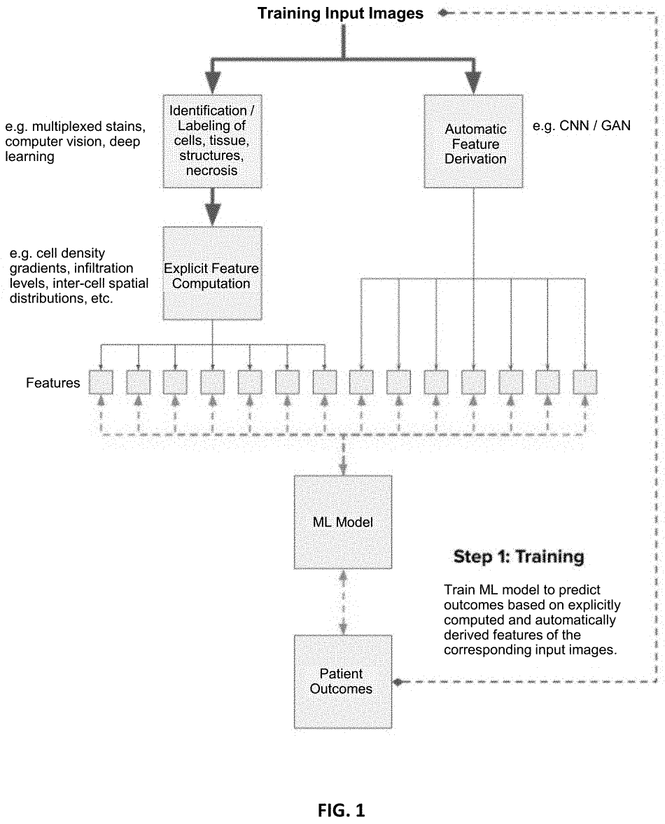

FIGS. 1 through 9 illustrate systems and methods of feature derivation and selection for assays leveraging tumor, immune cell and microenvironment characterization through machine learning based automated characterization. The following embodiments provide technical solutions and technical improvements that overcome technical problems, drawbacks and/or deficiencies in the technical fields involving machine learning optimization and training techniques that are limited to small feature sets with limited predictive power, as well as deficiencies in representations, visually and quantitatively of microenvironments, the inability to identify and incorporate intermediate outcomes, among other deficiencies in the area of prognosis prediction. As explained in more detail, below, technical solutions and technical improvements herein include aspects of improved combination of hand-engineered localization characteristics with complex machine learning-derived features may better predict immune activity and, in certain cases (e.g., malignant tissue), predict immune-mediated cell death, including the technical aspect of the optimization and integration of both hand-engineered and machine-derived features by machine learning, characterization of immune cell and microenvironments as volumes through three-dimensional virtual tissue modeling, and the incorporation of outputs from a predictive model as intermediate outcome proxies for training and feature selection, all provide technical advancements to the development and implementation of predictive systems employing characterizations of immune cells and microenvironments. Based on such technical features, further technical benefits become available to users and operators of these systems and methods. Moreover, various practical applications of the disclosed technology are also described, which provide further practical benefits to users and operators that are also new and useful improvements in the art.

FIG. 1 illustrates a flowchart of an illustrative methodology for training machine learning models for automatic feature detection for characterization of a microenvironment in accordance with one or more embodiments of the present disclosure.

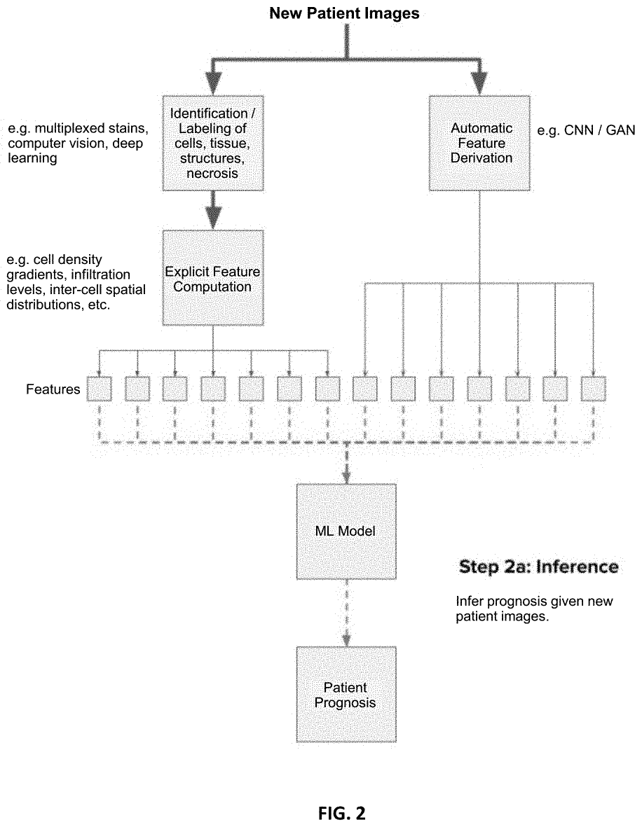

FIG. 2 illustrates a flowchart of an illustrative methodology for implementing machine learning models for automatic feature detection for characterization of a microenvironment in order to infer a patient prognosis using input images in accordance with one or more embodiments of the present disclosure.

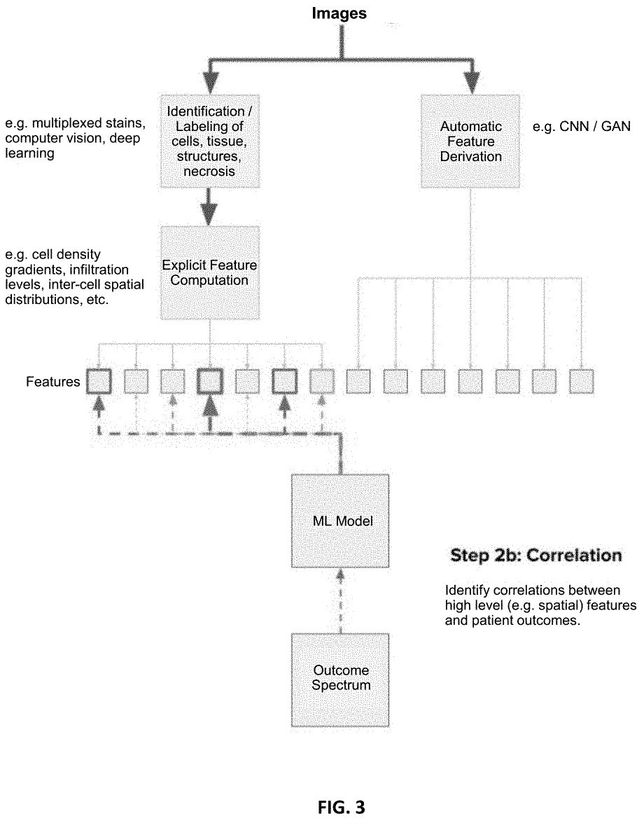

FIG. 3 illustrates a flowchart of an illustrative methodology for implementing machine learning models for determining correlations between detected features for characterization of a microenvironment and patient outcomes in accordance with one or more embodiments of the present disclosure.

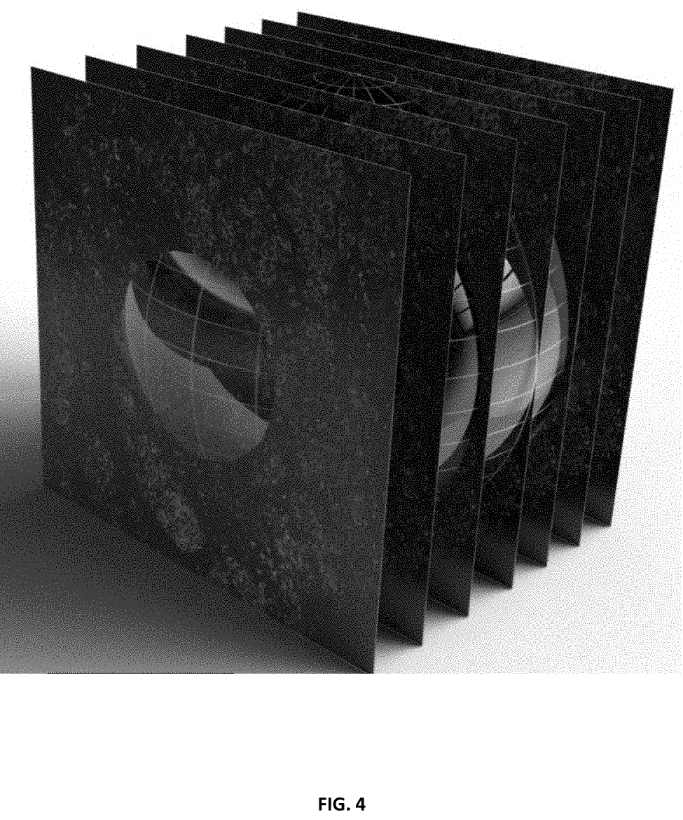

FIG. 4 is an illustration of tissue infiltration of target cells in three-dimensional stacked images as determined by the machine learning model approach described above in accordance with one or more embodiments of the present disclosure.

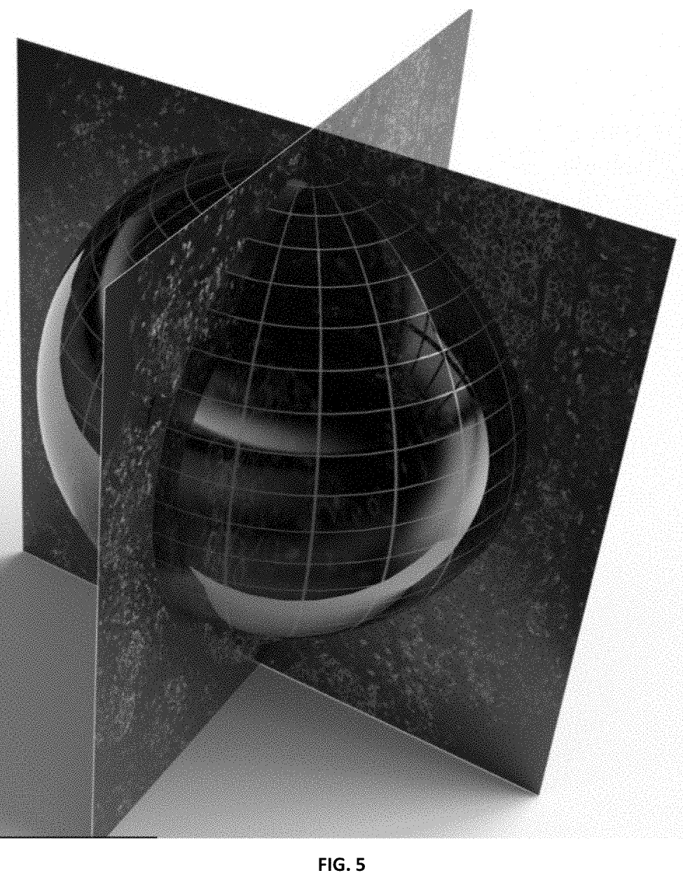

FIG. 5 is an illustration of More efficient extrapolation of distance of cells and structural volumes from tissue sections cut across divergent axes using the machine learning model approach described above in accordance with one or more embodiments of the present disclosure.

The presently disclosed embodiments relate to systems and methods for characterizing cells and microenvironments. In some embodiments, a tissue microenvironment approach characterizes the microenvironment of tissues, including the behaviors of immune cells. The tissue microenvironment system may ingest microenvironment input data and utilize machine learning models for feature selection to automatically determine microenvironment characterizations for predicting patient outcomes.

Embodiments of the present cell and microenvironment characterization system and method includes a novel approach to predict clinical and experimental outcomes by characterizing immune cells and their associated microenvironments through machine learning integration of hand-engineered and machine learning-derived complex features. This approach allows simultaneous analysis of the micro-environment of healthy and diseased tissue and the surrounding/interstitial immune cells, both spatially and temporally, to develop insights about cell interaction and movement relative to a variety of outcomes.

While immune cell quantification is currently the state of the art in terms of understanding cell-cell interactions, the present systems and methods provide a more nuanced and complete view of immune infiltration of tissues than existing methods. Previous measurements, such as immune cell density, do not geolocate immune cells and their corresponding microenvironments in relation to each other. Combining hand-engineered localization characteristics with complex machine learning-derived features may better predict immune activity and, in certain cases (e.g., malignant tissue), predict immune-mediated cell death.

For instance, geolocation via the present cell and microenvironment characterization system and method would allow understanding of migration patterns and rates of cell motility, possibly indicative of immune cells moving towards particular targeted diseased cells. In some embodiments, these techniques also allow for the geolocation of microvascularization in relation to the most relevant diseased cells and immune cells. While other analyses have shown that enumeration of CD4+ cells, CD8+ cells, macrophages, and others correlate with particular outcomes (e.g. lung, kidney, and breast cancer) there have not yet been analyses to show the geolocation of these cells relative to local angiogenesis, and its relevance.

In some embodiments, the optimization and integration of both hand-engineered and machine-derived features by machine learning is an additional unique aspect of the present methodology. This goes beyond existing approaches which principally correlate outcomes to single or small groups of hand-engineered features.

In some embodiments, by optimizing the cell and microenvironment characterization machine learning models for particular data sets with fine-tuning for specific applications, the platform can operate predictively to infer outcomes and/or secondary endpoints on new data. In addition, the use of machine learning integration of multiple features from fundamentally different originating sources creates an algorithmic robustness not seen in existing methodologies. This allows inference beyond identical input contexts to enable extrapolation to similar, but non-identical, settings.

In some embodiments, this algorithmic approach constitutes a previously undescribed synthetic analysis of immune cells and associated microenvironment contexts. This allows a capability unique and specific to embodiments of the present system: the use of outputs as intermediate outcome proxies for clinical/experimental outcomes in entirely novel settings. This capability is crucial to development of innovative therapies, such as engineered immune cells, for which there may be no existing data set with outcomes.

For example, in one such embodiment, the cell and microenvironment characterization methods may apply to brightfield or Hematoxylin and Eosin (H&E) stained serial tissue sections. Through virtual tissue modeling in three dimensions, immune cells and associated microenvironments are characterized as volumes (see, FIG. 4). This differs materially from existing approaches which have principally been two dimensional planar examinations of single tissue sections.

In an embodiment, biopsies of the liver or other organs can be analyzed to examine diseased or damaged tissue, through natural or toxicologic mechanisms, looking at histopathology to quantify infiltrating immune cells in the liver, and calculate many hand-calculated relative proximity measures or other derived relationship calculated between two or more of the following at a moment or in a time-series: immune cells, vascular structures, endothelial cells, hepatic cells, fatty changes to cells, steatosis and necrotic hepatic cells, fibrosis, or other structures.

In an embodiment, the detailed machine learning applied to toxicologic histopathology is used to determine patterns of pathology and correlate these with groupings in chemistry to infer causality of such disease or damage. One benefit of such groupings is to create predictive models that can be applied in silico to predict toxicological safety of candidate molecules which can be used to assess toxicology.

In such an embodiment, multiple features are integrated via machine learning such as:
a. Two dimensional micron-level distance of tumor-infiltrating lymphocytes (TIL) nuclei to tumor nest edges (see, FIG. 5);
b. Two or three dimensional distances between specific cell types/subtypes, diseased or damaged tissue, fatty changes, scarring/fibrosis, necrosis, vascular structures, ducts, and other anatomic structures;
c. Three dimensional distances between the membrane boundary of TIL cells to the membrane boundary of tumor cells;
d. Three dimensional calculated centers of TIL nuclei to calculated centers of tumor cells;
e. Three dimensional distance of membrane boundary or calculated centers of TIL nuclei to the edge of the vascular lumen;
f. Complex machine-derived features extracted from the center hidden layer of an auto-encoder architecture designed for high fidelity re-creation of inputs, where the complex features are high dimensional vectors (ranging from, e.g., 256 to 2048 in length depending on the input images) that are completely machine driven and data based and can be determined only by the parameters of the model and the data that is used for training;
g. Complex machine-derived features extracted from the terminal feature vectors of a volumetric convolutional architecture designed for mapping inputs to secondary cell health outcomes.

In some embodiments, the features may be ingested by one or more machine learning models to generate features for images of cells. For example, in some embodiments, basic features may be generated using Computer Vision-based methods include Scale-Invariant Feature Transform (SIFT), Speeded-Up Robust Features (SURF), Features from Accelerated Segment Test (FAST) that generate feature points in an image which can be used as feature vectors in a machine learning model. In addition or alternatively to these features, some embodiments may employ Unsupervised Convolutional Neural Networks which may be trained to generate the same image that is fed as an input via multiple encoder, decoder and convolutional layers. In this case, a U-Net based model or an incrementally down-sampling and then up-sampling CNN (Autoencoders) may be employed. Thus, the middle layer(s) serves as feature representation of the input image.

In some embodiments, the input images may include suitable images of tissue sections, such as microscopic imagery via a digital imaging device. In some embodiments, the tissue sections may include tissue of any suitable biological sample, such as, e.g., a human, cultured and/or animal subject, or other suitable biological sample having a microenvironment including cells.

Similarly, in some embodiments, a Supervised machine learning problem may be employed, such as, e.g., any CNN-based network like VGG, Inception, ResNet, U-Net or other CNN or any combination thereof, and use the layer before the output layer as a feature vector.

In some embodiments, multiple models may be employed for each features. For example, a combination of basic features, unsupervised CNNs and supervised CNNs may be employed for each feature.

The resulting evaluation of immune cells and associated microenvironment contexts are then utilized as in silico proxies for (1) immune cell activity quantification, (2) tumor angiogenesis (location and extent of vasculature), and (3) timeframe of apoptosis/cell death and/or proximity to solid tumor pyknotic tissue. This provides an intermediate outcome to potentially predict response to immunotherapy, measure disease progression, identify new staging and prognostic criteria, and guide treatment decisions such as angiogenesis-related treatment. Such an embodiment might then be iteratively improved through addition of other stains and multi-modal analysis such as genetic, expression, and epigenetic assessment localized at the cellular level.

Inputs

In some embodiments, the algorithms of the tissue microenvironment approach utilizes inputs from multiple modalities, individually or in groups. Examples of such inputs may include:
a. Live cells in various ex vivo presentations, such as in liquid media, solid media, in flow cytometry, in the context of tissues, or in organoids and simulated organs such as organ chips and systems;
b. Formalin fixed paraffin embedded tissue sections, in 2D or 3D (e.g., assembled from Z-stack imaging, serial sections, MRI, CT, etc.);
c. Frozen pathology sections, in 2D or 3D (e.g., assembled from Z-stack imaging, serial sections, MRI, CT, etc.);
d. In vitro engineered cells;
e. Sourced or transplanted autologous or allogeneic cells;
f. Spatial sequencing of cells to identify genetic sequences localized to specific microscopic regions such as FISSEQ and Slide-Seq.

In some embodiments, a particular measure may employ unstained or H&E based stains, but may also incorporate other stains, such as immunohistochemistry (IHC) for specific lymphocyte subsets, single-cell expression, genomic or epigenetic data, among other suitable data inputs.

Images of one or more of the above inputs may be supplied to the system for implementing the microenvironment characterization approach. Any other suitable inputs for feature selection in medical or pathological imagery be employed.

### Feature Selection

In some embodiments, the approach may characterize a variety of target cell types with respect to possible patient conditions or outcomes. In some embodiments, the target cell types below may be characterized as cancer cells or immune cells, but in some embodiments, the target cells or tissues may originate from samples from patients with autoimmune disease, contused, damaged tissues or infections. From training data, and then when making predictions on new data, a system of the present approach may automatically or manually select and tune feature selection techniques. Feature selection may be effectuated using one or more hand-engineered features and/or machine-derived features.

### Hand-Engineered Feature Selection

In some embodiments, hand-engineered features may include an individual or combination of features characterizing the microenvironment. In some embodiments, the hand-engineered features may be incorporated as the only feature selection technique, however, in some embodiments, the hand-engineered features are algorithmically combined with or replaced by derived compound features. In some embodiments, the hand-engineered features may include, e.g., human annotations of microenvironment imagery, algorithmic measurement and/or computation according to microenvironment imagery, or any suitable combination thereof to create features describing structures, positions and behaviors of microenvironments in microenvironment imagery.

In one embodiment, a hand-engineered feature may include infiltration depth of immune cells into the tumoral boundary that is calculated algorithmically or manually. Infiltration depth is a novel concept as a feature of microenvironment characterization. In some embodiments, infiltration depth reflects the extent to which the tumor is being penetrated by the immune system. In the case of solid tumors, infiltration depth may be used as a metric of the efficacy score of the mounted immune response where greater infiltration depth indicates a more effective immune response.

In some embodiments, a tumoral boundary can be calculated via individual classification of cell types, and constructing a concave hull algorithm for determining tumoral boundary. Parameters of cell classification and concave hull algorithm are adjustable by the system to optimally fit training data. Distance to tumoral boundary can be determined via one of several methods, including (a) distance to nearest neighbor determined via expanding circle algorithm, (b) shortest distance by calculating the distance from the cell to the normal of the boundary or, if such normal does not intersect with the cell, the distance to the closest vertex, or (c) selecting the shortest distance from a precalculated table of potential nearest neighbors. Each of these distance calculations can be performed from the centroid of a cell, surface of a cell, or arbitrary position relative to the cell to the surface of the boundary or some arbitrary distance or position relative to the boundary. Additionally, distance can be calculated in multiple dimensions by extending the expanding circle technique to use a sphere or n-sphere in the case of n-dimensional solution space or by computing the distance of the normal of the boundary surface (in 2-D, 3-D, or n-D) to the cell.

In one embodiment, a hand-engineered feature may include an aggregate measure of the level of immune infiltration, calculated from the sum, average, median, or mode of distance of a target cell(s) (e.g., cancer cell or pathogen) to the closest immune cell, or any combination thereof.

In one embodiment of an engineered feature, the past or future movement and/or locations of immune cells is predicted from the gradient in the density of individual algorithmically or manually identified cells and structures (such as tumor boundaries, organ/tissue boundaries, blood/lymph vessels) across a sample or subset of a sample, or a larger image composed of smaller stitched images. Movement may be inferred from machine learning analysis of time series images based on changes in the gradient and/or density between prior and current images, expert analysis based on context, and/or a combination of density, spatial distribution and structure. This movement may be used to measure the progress of immune infiltration based on distance from the source of the gradient, among other applications.

In some embodiments, movement inferencing may be performed with a suitable machine learning model to infer the movement of cells and/or cell structures. In some embodiments, a machine learning model may be employed to infer a direction of movement of cells. For example, sequential images may be used to generate ground truth data and train a CNN-based network against detected directions of cell movement in the ground truth data. Directions may include, e.g., up, down, left, right, top-right, top-left, bottom-right, bottom-left, etc. In some embodiments, the CNN-based network may include, e.g., VGG, ResNet, Inception, etc.

In some embodiments, a machine learning model may be employed to infer a next position of cells. For example, sequential images may be used to generate ground truth data and train a three-dimensions (3D) CNN-based network against detected positions of cells in the ground truth data. In some embodiments, the CNN-based network may include, e.g., custom trained 3D versions of VGG, ResNet, Inception, etc. In some embodiments, the machine learning model may instead or in addition use recurrent neural network (RNN)-based models and/or generative adversarial networks (GAN).

In one embodiment of an engineered feature, utilizing multiplexed IHC, in-situ hybridization (ISH), single cell in situ sequencing, area-based sequencing, spatial sequencing, or other modalities assays in addition to or sequentially (before or after bleaching/washing) of a secondary brightfield (e.g. hematoxylin and eosin) or other secondary sensing method to automatically train an automatic feature derivation system that generates features and uses them in combination with hand engineered features for further use (classification or feature correlation) to recognize components, features, cell types and subtypes and other characteristics depicted by said IHC, ISH, single cell sequencing, area-based sequencing, spatial sequencing, or other assays solely through the secondary sensing method.

In one embodiment, a hand-engineered feature may include immune cell activity/efficacy (e.g., an efficacy score) inferred from proximity to evidence of tumor autophagy, apoptosis, necrosis, or other indicators of immune cell activity such as degranulation, chemokine signaling or other morphological changes associated with immune activation.

In one embodiment, automated or manual spatial characterization of individually identified cells and structures including but not limited to tumoral boundaries, organ locations, blood and lymph vessels coordinates of cell locations using a whole body anatomical coordinate system. This coordinate system can be characterized by a specific reference point in the body or standard coordinate systems such as those defined by the intersection of axial, coronal and sagittal (midsagittal) anatomical planes. The coordinate system location of the imaged sample is captured during the time of biopsy and individual cell and structure locations within the sample are derived from this biopsy anatomical coordinate system location.

In some embodiments, hand-engineered features may be determined. The term “hand-engineered” refers to explicitly designed measurements, e.g., algorithmic features as opposed to machine learned (e.g., via one or more machine learning models). Accordingly, the hand-engineered features may be manually measured or algorithmically determined according to the explicitly designed measurements.

In one embodiment, a hand-engineered feature may include characterizing a target cell and surrounding cell status using brightfield microscopy, IHC, and/or FISH including cell health/death status, tissue damage, and other biomarkers.

In one embodiment, a hand-engineered feature may include differentiation of sub-classes of immune cells, such as CD8+ T-cells, CD4+ T cells, Macrophages, NK cells, etc.

In one embodiment, a hand-engineered feature may include distance to closest blood vessel, or proxy assays for distance to blood vessels, such as waste products, gases, vesicles, etc.; algorithm parameters for determining blood vessel/structure location and classification can be automatically tuned by system.

In one embodiment, a hand-engineered feature may include distance to an external absolute point of reference in the body, organ or tissue. In cases where an absolute point of reference is provided, the absolute distance, coordinates, types and characteristics of individual cells can be learned by the system and then predicted on future samples.

In one embodiment, a hand-engineered feature may include the distance of these cells to local microvascularization (e.g., tumor angiogenesis) and its relevance.

In one embodiment, a hand-engineered feature may include the annotation of ductal structures (e.g., cellular and/or physiological structures pertaining to ducts) and/or the distance of these cells to local ductal structures.

In one embodiment, a hand-engineered feature may include physical immune cell or target cell features such as blebs, or other morphology, in order to assess cell motility.

In one embodiment, a hand-engineered feature may include intersection of immune cell infiltration indicating potential cancer stem or progenitor cells.

In one embodiment, a hand-engineered feature may include cell motility inferred from distance or movement of a population from a tumoral border or blood vessel.

In some embodiments, when time sequence data (e.g., video or time lapse) is available, a hand-engineered feature may include tracking rates of motility for individual cells from a relative or absolute reference point. This may apply to sequences of cells from live cell imaging, ex vivo studies, and liquid biopsy data (detecting proximity of cancer and immune cells in blood samples or flows. In some embodiments, the time sequence data may include blocks of subsequent images fed to, e.g., a 3D CNN-based model trained to detect cell movement and motility as described above.

In some embodiments, when multiple serial slides or depth data may be available, a hand-engineered feature may include combining slides into a 3D model of tissue, additionally encompassing multiple z-stacked (refocused) images taken from individual slide sections. When multiple sides are available but individual serial sections are either missing or of an incompatible type (e.g. a different stain), extrapolation between serial sections allows for compensation of this deficit. (FIG. 4). In another variation (FIG. 5), tissue sections may be created across divergent axis and reassembled algorithmically to more rapidly estimate locations and volumes due to a much lower computational burden per cubic unit of volume compared to serial slices along the same axis.

Derived Compound Feature Selection

As described above, the hand-engineered features may be supplemented by or replaced by derived compound features. In some embodiments the derived compound features are determined using one or more machine learning models. The machine learning models may include, e.g., a suitable classifier for feature selection tasks. For example, in some embodiments, the feature selection models may include, e.g., convolutional neural networks (CNN) and/or generative adversarial networks (GAN), however other models may be employed, such as, e.g., random forest classification, naive Bayes, autoencoder classification, or other suitable classifiers. The derived compound features may be derived from one or more machine learning models. In some embodiments, each derived compound features is derived from a respective machine learning. However, in some embodiments, multiple derived compound features may be derived from a single machine learning model. Depending on the problem or availability of data, multiple models may be sued for generating different features. For example, if the data is not labeled, then using Autoencoder based models, trained adversarially or non-adversarially, may be the best approach.

In some embodiments, derived compound features may include preprocessing of inputs to understand the inter-feature changes for generalization.

In some embodiments, derived compound features may include a use of hidden representation layers in an auto-encoder architecture which processes inputs for optimal reconstruction.

In some embodiments, derived compound features may include a use of feature vectors as described above in a convolutional neural network architecture which predicts outcomes from inputs.

In some embodiments, derived compound features may include combining traditional computer vision (e.g., Computer vision techniques such as segmentation, color separation, fourier transforms, feature detection can be used to increase the feature richness) and data science methods along with state of the art machine learning and deep learning algorithms. In some embodiments, the general approach is to provide as many features as possible to the training model and let the model figure out the most useful features.

In some embodiments, derived compound features may include a supervised learning of data gathered over time for time-series based prediction to generate features representative of the entire data including current and prior data from current and prior images.

In some embodiments, derived compound features may include creating additional data points by learning generative models that understand the underlying data patterns. In some embodiments, generative models can include virtual staining to better understand the morphology of the cells. Some of the virtual stains like IHC may be better indicative of some things as opposed to HE stains.

In some embodiments, derived compound features may include using multimodal models that combine information from different data types for accurate predictions and feature generation.

In some embodiments, derived compound features may include using feature correlations to find and eliminate repeated features (“repeat feature reduction”).

In some embodiments, derived compound features may include combining multiple models or features and weighting them with respect to the output, creating an ensemble model.

In some embodiments, derived compound features may include validate and test the models using statistical tools, visualizations, graphs, feature maps for analysis.

In some embodiments, derived compound features may include Gradient Class Activation Maps to visualize areas of interest that influence the decision, also helps humans to focus on the important parameters. In some embodiments, the areas of interest may be model dependent. In some embodiments, the areas of interest may visually show on what the model bases its decision. In some embodiments, more complex features which are derived through training can show which patch of the image was most influential in resulting a particular decision.

Embodiments of the present invention may include any other derived feature selection techniques, using, e.g., machine learning algorithms suitable for extracting features from medical imagery, including the inputs described above.

### Outcomes

In some embodiments, training of the models used for characterization and prognosis prediction may utilize the inputs described above and may be trained against multiple patient outcomes. For example, the patient outcomes used may include:
a. Overall response rate;
b. Durability of response;
c. Duration of remission/time to relapse;
d. Progression free survival;
e. Overall survival.
f. In some embodiments, intermediate patient outcomes may also be employed. For example, some intermediate patient outcomes may include:
g. Histologic features labeled by pathologists;
h. Degree of differentiation of immune cells;
i. Live-dead cell assays;
j. Inflammation assays;
k. Apoptosis assays;
l. Cell proliferation assays;
m. Cell motility assays;
n. Cell viability and toxicity assays;
o. Mitochondrial membrane potential assays;
p. Oxidative stress assays, such as electron-transfer and hydrogen atom mediated assays;
q. Absorption, distribution, metabolism, and elimination assays;
r. Reactive oxygen species assays.

In some embodiments, there may be various approaches to utilizing outcome data, either separately or in combination. For example, in some embodiments, for data sets with a large number of outcome cases, feature derivation and selection models will be trained end-to-end directly on the outcome variables. For data sets with a small number of outcomes cases, some embodiments may include large data sets with outcomes in related diseases would be used for pre-training, with subsequent transfer learning by fine-tuning for the specific disease

For data sets with no outcomes, some embodiments may include large data sets with outcomes in related diseases used for all phases of training. Derived and selected features would then be used to predict likely outcomes for the original data set.

For data sets with no outcomes and no related data sets with outcomes, some embodiments may include unsupervised machine learning to analyze principal components and organize features into clusters. Inter-cluster and intra-cluster analysis will then determine feature sets that are representative of various characterizations of immune cells.

However, in some embodiments, all cases may derive complex features and features selected by models will also be used as intermediate proxy outcomes for immune cell activity. For instance, bioreactor settings can be adjusted such that resulting clonal expansion generates cells that better match such features.

### Usefulness

In some embodiments, the machine learning algorithm may be employed for a variety of use cases where characterizing microenvironments is beneficial. Using training data sets, the machine learning models for inferring prognosis of a patient may be trained for particular use cases. Training may be performed by comparing the prognoses inferred by the prognosis inference models to known outcomes associated with input images. Such comparison can include the determination of loss using a suitable loss or optimization function. The loss may be backpropagated to the prognosis inference models to update model parameters and weights thereof using, e.g., simple gradient descent, or other suitable backpropagation algorithm (see, FIG. 1).

In some embodiments, the systems and methods for implementing machine learning models for cell and microenvironment characterization for prognosis predictions may include the use of machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network or other suitable network. The machine learning techniques for cell and microenvironment characterization may include regression or classification models employing one or more of the techniques described above. In some embodiments and, optionally, in combination of any embodiment described above or below, an exemplary implementation of Neural Network may be executed as follows:
a. define neural network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values, functions and aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the trained prognosis inference models may employ the features extracted from the input images to infer patient prognosis based on the patient outcome training described above. Thus, a new image may be provided to the system, as shown in FIG. 2, at which point the feature algorithms and/or inputs may extract a combination of engineered features and derived compound features. The prognosis inference model of the system may ingest the extracted features and infer a prognosis of the patient associated with the image based on the training.

For example, uses may include clinical grading of tumors for invasiveness and patient prognosis. In some embodiments, to use the microenvironment characterizations to grade tumors, the machine learning model may be customized by training with data sets for each tumor type including prognosis outcomes with respect to the data sets.

In some embodiments, the microenvironment characterization may include organization and classification of patients for clinical trials based on predicted patient response and suitability. In some embodiments, to use the microenvironment characterizations to organize and classify patients for clinical trials may include training using training data sets with prognosis outcomes for similar diseases

In some embodiments, the microenvironment characterization may include objective, reproducible evaluation of immune cell activity and microenvironments for evaluation of therapeutic effectiveness. In some embodiments, to use the microenvironment characterizations to evaluate immune cell activity and microenvironments for therapeutic effectiveness can include training using training data sets with immune activity outcomes and/or fine-tuning data sets with therapeutic outcomes

In some embodiments, the microenvironment characterization may include optimization of methods, settings, and systems for engineering of immune cell therapeutics. In some embodiments, to use the microenvironment characterizations to optimize immune cell therapeutics can include training using training data sets with cell engineering methods, settings, and systems inputs, and training data sets including prognosis outcomes for the disease the therapeutic is intended for

In some embodiments, the microenvironment characterization may include discovery of predictive features of immune cell activity through machine learning analysis of the output of the prognosis prediction, independently of existing methods. In some embodiments, to use the microenvironment characterizations to discover and predict immune cell activity can include training using training data sets with prognosis outcomes for each tumor type.

In some embodiments, the microenvironment characterization may include informing research by visualization of cellular interactions, such as through heat maps, gradient maps, and other visualizations of critical features. In some embodiments, to use the microenvironment characterizations to visualize cellular interactions can include creating graphical representation software units specific to the research needs of the users.

In some embodiments, the microenvironment characterization may include evaluating immune responses to infectious diseases such as, e.g., COVID-19, influenzas, Ebola, HIV, among others. In some embodiments, to use the microenvironment characterizations to predict immune response for infectious disease can include training using training data sets including disease resolution outcomes for each infection type.

In some embodiments, the microenvironment characterization may include xenotransplantation in human and veterinary medicine. In some embodiments, to use the microenvironment characterizations to predict xenotransplantation may include model training with training data sets with transplantation outcome for each cross-species pair.

In some embodiments, the microenvironment characterization may include autoimmunity prognosis prediction, such as, with, atherosclerosis, rheumatoid arthritis, among others. In some embodiments, to use the microenvironment characterizations to predict autoimmunity prognosis may include training using training data sets with patient outcomes for each disease.

In some embodiments, the microenvironment characterization may include detection of minimal residual disease in liquid tumors. In some embodiments, to use the microenvironment characterizations to detect the minimal residual disease may include training using training data sets with prognosis outcomes for each liquid cancer type.

In some embodiments, the microenvironment characterization may include differentiating between autophagy, apoptosis and necrosis. In some embodiments, to use the microenvironment characterizations to differentiate by autophagy, apoptosis and necrosis may include training using training data sets with assay indicators of cell death course for each cell.

In some embodiments, the microenvironment characterization may include nuclear structures of a tumor that could be an indicator of prognosis. In some embodiments, to use the microenvironment characterizations to employ nuclear structures of tumors to indicate prognosis may include training using training data sets with prognosis outcomes for each tumor type.

In some embodiments, the microenvironment characterization may include neuroanatomy and cell structure. In some embodiments, to use the microenvironment characterizations to evaluate neuroanatomy and cell structure may include training using training data sets with neurological ground truth labeling for different organ contexts.

In some embodiments, the microenvironment characterization may include analyzing circulating tumor cells that cause metastasis. In some embodiments, to use the microenvironment characterizations to assess metastasis using circulating tumor cells may include training using training data sets with assay indicators of immune cell sub-typology.

In some embodiments, the prognosis inference machine learning models may also be employed to determine correlations between individual hand-engineered features and patient outcomes, as shown in FIG. 3, to identify features that have greater influence on patient outcomes. Correlations are determined by generative machine learning models that discover maximal activation profiles for specific patient outcomes. Accordingly, the system may automatically produce weightings in feature selection to reflect the influence of each feature.

In some embodiments, the inferred prognosis may then be provided to the user via a display of a computing device. For example, the prognosis may be sent to the computing device, e.g., as a notification, an alert, a user interface component, etc. In some embodiments, the prognosis may include a text description, an image and/or graphic depicting the prognosis or any other form of representing the prognosis or any combination thereof. In some embodiments, the inferred prognosis may be displayed directly on a display of the system upon output of the inferred prognosis by the prognosis inferencing machine learning models.

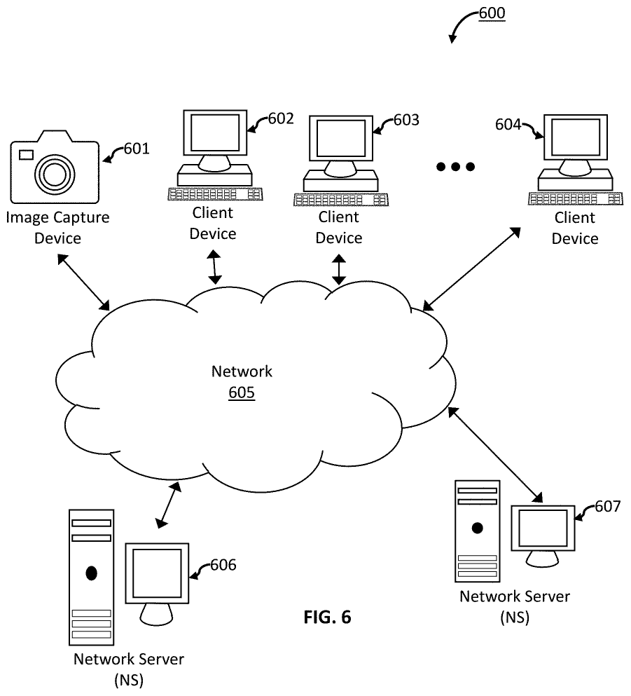

FIG. 6 depicts a block diagram of an exemplary computer-based system and platform 600 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 600 may be configured to manage a large number of members and concurrent transactions, as detailed herein. In some embodiments, the exemplary computer-based system and platform 600 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 6, members 602-604 (e.g., clients) of the exemplary computer-based system and platform 600 may include virtually any computing device capable of receiving and sending a message over a network (e.g., cloud network), such as network 605, to and from another computing device, such as servers 606 and 607, each other, and the like. In some embodiments, the member devices 602-604 may be personal computers, multiprocessor systems, microprocessor-based or programmable consumer electronics, network PCs, and the like. In some embodiments, one or more member devices within member devices 602-604 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 602-604 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 602-604 may run one or more applications, such as Internet browsers, mobile applications, voice calls, video games, videoconferencing, and email, among others. In some embodiments, one or more member devices within member devices 602-604 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 602-604 may be specifically programmed by either Java, .Net, QT, C, C++ and/or other suitable programming language. In some embodiments, one or more member devices within member devices 602-604 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, image capture device 601 may be included to capture and communicate imagery, such as, e.g., medical imagery including assays, brightfield or H&E stained microscopy, Z-stack imaging, serial sections, MRI, CT, etc. In some embodiments, the image capture device 601 may include a wired or wireless connection to the network 605 for automatic communication of digital imagery to member device 602-604 and/or servers 606 and 607. However, in some embodiments, the image capture device 601 may be separate from the network 605 and the imagery is scanned or otherwise reproduced at a member device 602-604 or server 606 and 607 to load the imagery into embodiments of the present immune cell and microenvironment characterization models. In some embodiments, these models may be implemented on one or more of the member device 602-604 and/or servers 606 and 607, including feature selection, modelling, outcome prediction, and feature-to-outcome correlation as described in more detail above.

In some embodiments, the exemplary network 605 may provide network access, data transport and/or other services to any computing device coupled to it. In some embodiments, the exemplary network 605 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 605 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 605 may include and implement, as an alternative or in conjunction with one or more of the above, a WiMAX architecture defined by the WiMAX forum. In some embodiments and, optionally, in combination of any embodiment described above or below, the exemplary network 605 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 605 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 605 may also include mass storage, such as network attached storage (NAS), a storage area network (SAN), a content delivery network (CDN) or other forms of computer or machine readable media.

In some embodiments, the exemplary server 606 or the exemplary server 607 may be a web server (or a series of servers) running a network operating system, examples of which may include but are not limited to Microsoft Windows Server, Novell NetWare, or Linux. In some embodiments, the exemplary server 606 or the exemplary server 607 may be used for and/or provide cloud and/or network computing. Although not shown in FIG. 6, in some embodiments, the exemplary server 606 or the exemplary server 607 may have connections to external systems like email, SMS messaging, text messaging, ad content providers, etc. Any of the features of the exemplary server 606 may be also implemented in the exemplary server 607 and vice versa.

In some embodiments, one or more of the exemplary servers 606 and 607 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 602-604.

In some embodiments and, optionally, in combination of any embodiment described above or below, for example, one or more exemplary computing member devices 602-604, the exemplary server 606, and/or the exemplary server 607 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

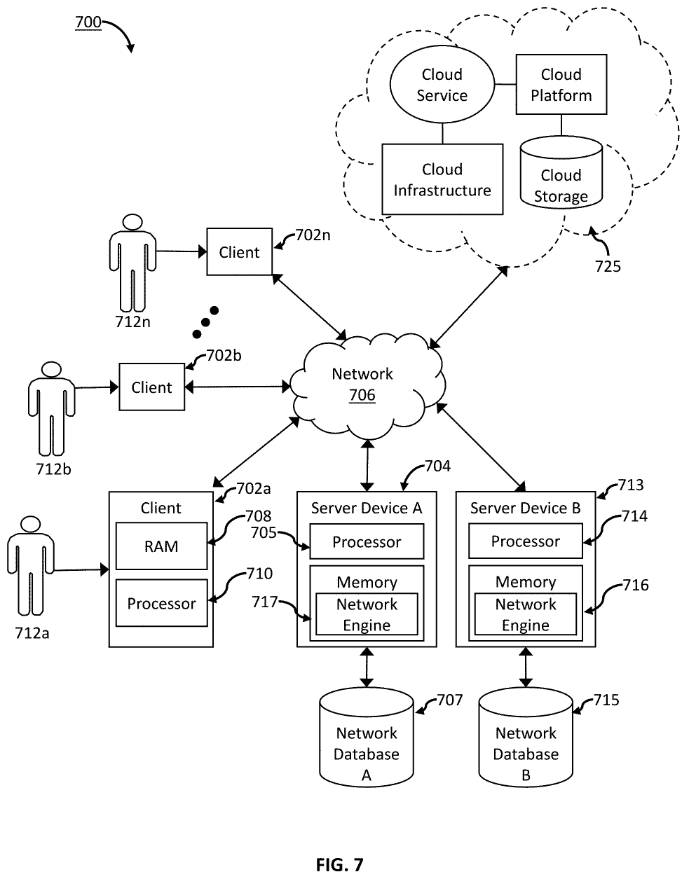

FIG. 7 depicts a block diagram of another exemplary computer-based system and platform 700 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 702a, 702b through 702n shown each at least includes a computer-readable medium, such as a random-access memory (RAM) 708 coupled to a processor 710 or FLASH memory. In some embodiments, the processor 710 may execute computer-executable program instructions stored in memory 708. In some embodiments, the processor 710 may include a microprocessor, an ASIC, and/or a state machine. In some embodiments, the processor 710 may include, or may be in communication with, media, for example computer-readable media, which stores instructions that, when executed by the processor 710, may cause the processor 710 to perform one or more steps described herein. In some embodiments, examples of computer-readable media may include, but are not limited to, an electronic, optical, magnetic, or other storage or transmission device capable of providing a processor, such as the processor 710 of client 702a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 702a through 702n may also comprise a number of external or internal devices such as a mouse, a CD-ROM, DVD, a physical or virtual keyboard, a display, or other input or output devices. In some embodiments, examples of member computing devices 702a through 702n (e.g., clients) may be any type of processor-based platforms that are connected to a network 706 such as, without limitation, personal computers, digital assistants, personal digital assistants, smart phones, pagers, digital tablets, laptop computers, Internet appliances, and other processor-based devices. In some embodiments, member computing devices 702a through 702n may be specifically programmed with one or more application programs in accordance with one or more principles/methodologies detailed herein. In some embodiments, member computing devices 702a through 702n may operate on any operating system capable of supporting a browser or browser-enabled application, such as Microsoft™ Windows™, and/or Linux. In some embodiments, member computing devices 702a through 702n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 702a through 702n, users, 712a through 712n, may communicate over the exemplary network 706 with each other and/or with other systems and/or devices coupled to the network 706. As shown in FIG. 7, exemplary server devices 704 and 713 may be also coupled to the network 706. In some embodiments, one or more member computing devices 702a through 702n may be mobile clients.

In some embodiments, at least one database of exemplary databases 707 and 715 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

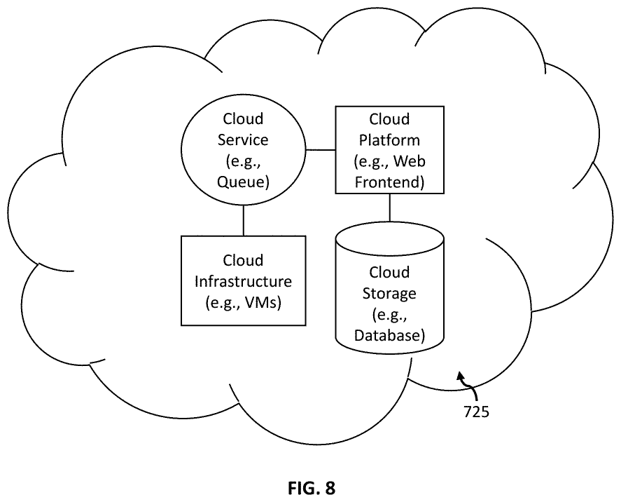

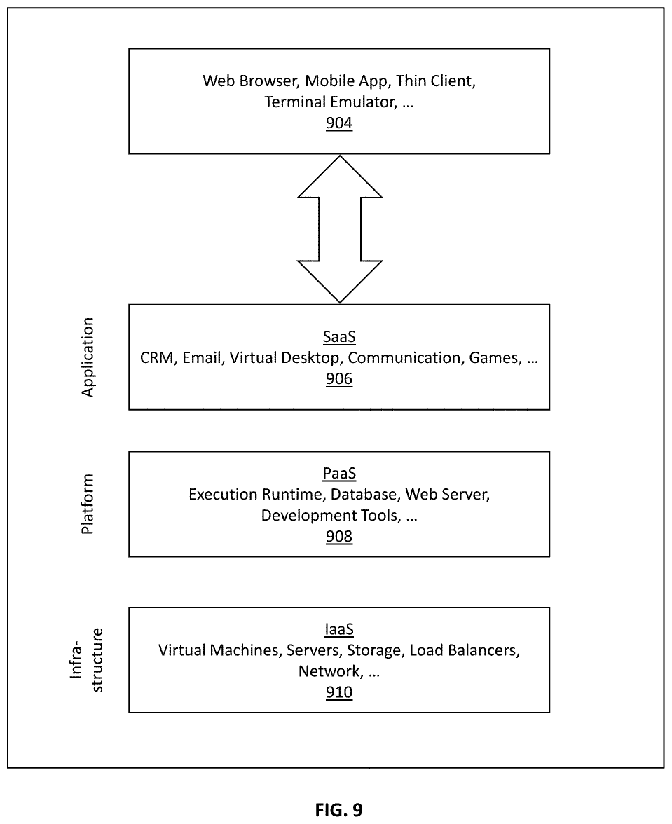

In some embodiments, the exemplary inventive computer-based systems/platforms, the exemplary inventive computer-based devices, and/or the exemplary inventive computer-based components of the present disclosure may be specifically configured to operate in a cloud computing/architecture 725 such as, but not limiting to: infrastructure a service (IaaS) 910, platform as a service (PaaS) 908, and/or software as a service (SaaS) 906 using a web browser, mobile app, thin client, terminal emulator or other endpoint 904. FIG. 8 and FIG. 9 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RCS, CAST and Skipjack), cryptographic hash algorithms (e.g., MD5, RIPEMD-160, RTR0, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative and not restrictive.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
receiving, by at least one processor, at least one microenvironment image from a microscopic imaging device;
receiving, by the at least one processor, at least one engineered microenvironment feature for each microenvironment image of the at least one microenvironment image;
utilizing, by the at least one processor, at least one feature extraction machine learning model to extract at least one derived compound microenvironment feature from each microenvironment image of the at least one microenvironment image;
utilizing, by the at least one processor, at least one prognosis inference machine learning model to infer a prognosis associated with the at least one microenvironment image using the at least one engineered microenvironment feature and the at least one derived compound microenvironment feature; and
generating, by the at least one processor, a notification indicating the prognosis based on the at least one microenvironment image for display on a user computing device in communication with the at least one processor.

2. The method of claim 1, further comprising:
identifying, by the at least one processor, cellular types and subtypes of cells in the at least one microenvironment image;
determining, by the at least one processor, individual cell distances of between the cells according to the cellular types and subtypes of the cells; and
determining, by the at least one processor, an engineered microenvironment feature comprising at least one aggregate measure of cell distance to represent the individual cell distances an aggregate.

3. The method of claim 1, further comprising:
determining, by the at least one processor, locations of immune cells in the at least one microenvironment image;
determining, by the at least one processor, at least one gradient associated with changes in density of the immune cells based at least in part on the locations of the immune cells;
determining, by the at least one processor, at least one change in the at least one gradient from at least one prior gradient of at least one prior microenvironment image; and
extrapolating, by the at least one processor, an engineered microenvironment feature comprising future immune cell positions based at least in part on the at least one change in the at least one gradient.

4. The method of claim 1, further comprising:
identifying, by the at least one processor, immune cell activity of immune cells based on morphological changes associated with immune activation between the at least one microenvironment image and at least one prior microenvironment image;
determining, by the at least one processor, an efficacy score of each immune cell from at least one immune cell based on the immune cell activity; and
determining, by the at least one processor, an engineered microenvironment feature comprising an aggregate immune cell efficacy score based on a statistical aggregation of the efficacy score of each immune cell.

5. The method of claim 1, further comprising:
identifying, by the at least one processor, immune cells and vascular structures in the at least one microenvironment image; and
determining, by the at least one processor, an engineered microenvironment feature comprising a distance between the immune cells and the vascular structures.

6. The method of claim 1, wherein the at least one engineered microenvironment feature comprises at least one of:
aggregate immune cell infiltration;
immune cell sequencing;
diseased or damaged tissue;
steatosis or fatty changes;
scarring and fibrosis;
necrosis;
vascular structures;
ductal structures;
individual cell spatial characterization;
target cell and surrounding cell status;
differentiation of sub-classes of immune cells;
cell morphology;
an intersection of immune cell infiltration;
cell motility;
rates of motility of individual cells;
other structures; or
combinations thereof.

7. The method of claim 1, wherein the at least one derived compound microenvironment feature comprises at least one of:
inter-feature changes;
auto-encoder reconstruction;
convolutional neural network feature vectors;
computer vision output;
deep learning output;
time-series based prediction of features representative of a time-series of images;
data points produced by generative models;
features generated from multi-modal modelling of image data;
repeat feature reduction;
an ensemble model of weighted of features;
gradient class activation maps; and
combinations thereof.

8. The method of claim 1, further comprising:
comparing, by the at least one processor, the prognosis with a known prognosis determine a loss; and
backpropagating, by the at least one processor, the loss to the at least one prognosis inference machine learning model to update parameters of the at least one prognosis inference machine learning model.

9. The method of claim 1, further comprising determining, by the at least one processor, a correlation between patient outcomes and each engineered microenvironment feature of the at least one engineered microenvironment feature.

10. The method of claim 1, further comprising:
determining, by the at least one processor, a correlation between biological sample outcomes and each engineered microenvironment feature of the at least one engineered microenvironment feature;
utilizing, by the at least one processor, at least one toxicologic histopathology machine learning model to determine patterns of pathology to classify the correlation with groupings in chemistry based at least in part on the correlation between the biological sample outcomes to infer causality; and
generating, by the at least one processor, at least one predictive model based at least in part on the groupings to predict toxicological safety.

11. A method comprising:
receiving, by at least one processor, at least one microenvironment image captured by an imaging device and depicting a cellular microenvironment of a biological sample;
receiving, by the at least one processor, at least one structure identifier in the at least one microenvironment image via user selection to identify at least one structure in the cellular microenvironment;
determining, by the at least one processor, at least one engineered microenvironment feature for each microenvironment image of the at least one microenvironment image based at least in part on the at least one structure identifier and at least one feature computation;
utilizing, by the at least one processor, at least one feature extraction machine learning model to extract at least one derived compound microenvironment feature from each microenvironment image of the at least one microenvironment image based on trained feature extraction parameters of the at least one feature extraction machine learning model and each microenvironment image of the at least one microenvironment image;
utilizing, by the at least one processor, at least one prognosis inference machine learning model to infer a prognosis associated with the biological sample based on trained prognosis inference parameters of the at least one prognosis inference machine learning model and the at least one engineered microenvironment feature and the at least one derived compound microenvironment feature; and
generating, by the at least one processor, a notification indicating the prognosis based on the at least one microenvironment image for display on a user computing device in communication with the at least one processor.

12. The method of claim 11, wherein the at least one feature extraction machine learning model comprises at least one convolutional neural network trained to ingest the at least one microenvironment image and output a set of annotations representing the at least one derived compound microenvironment feature.

13. The method of claim 11, wherein the at least one feature extraction machine learning model comprises at least one generative adversarial network trained to ingest the at least one microenvironment image and output a set of annotations representing the at least one derived compound microenvironment feature.

14. The method of claim 11, further comprising:
identifying, by the at least one processor, cellular types and subtypes of cells in the at least one microenvironment image;
determining, by the at least one processor, individual cell distances of between the cells according to the cellular types and subtypes of the cells; and
determining, by the at least one processor, an engineered microenvironment feature comprising at least one aggregate measure of cell distance to represent the individual cell distances an aggregate.

15. The method of claim 11, further comprising:
identifying, by the at least one processor, immune cell activity of immune cells based on morphological changes associated with immune activation between the at least one microenvironment image and at least one prior microenvironment image;
determining, by the at least one processor, an efficacy score of each immune cell from at least one immune cell based on the immune cell activity; and
determining, by the at least one processor, an engineered microenvironment feature comprising an aggregate immune cell efficacy score based on a statistical aggregation of the efficacy score of each immune cell.

16. The method of claim 11, further comprising:
identifying, by the at least one processor, immune cells and vascular structures in the at least one microenvironment image; and
determining, by the at least one processor, an engineered microenvironment feature comprising a distance between the immune cells and the vascular structures.

17. The method of claim 11, further comprising:
comparing, by the at least one processor, the prognosis with a known prognosis to determine a loss; and
backpropagating, by the at least one processor, the loss to the at least one prognosis inference machine learning model to update parameters of the at least one prognosis inference machine learning model.

18. The method of claim 11, further comprising determining, by the at least one processor, a correlation between patient outcomes and each engineered microenvironment feature of the at least one engineered microenvironment feature.

19. The method of claim 11, further comprising:
determining, by the at least one processor, a correlation between biological sample outcomes and each engineered microenvironment feature of the at least one engineered microenvironment feature;
utilizing, by the at least one processor, at least one toxicologic histopathology machine learning model to determine patterns of pathology to classify the correlation with groupings in chemistry based at least in part on the correlation between the biological sample outcomes to infer causality; and
generating, by the at least one processor, at least one predictive model based at least in part on the groupings to predict toxicological safety.

20. A non-transitory computer readable medium having software instruction stored thereon, the software instructions configured to cause at least one processor of at least one computer to perform steps to:
receive at least one microenvironment image from a microscopic imaging device;
receive at least one engineered microenvironment feature for each microenvironment image of the at least one microenvironment image;
utilize at least one feature extraction machine learning model to extract at least one derived compound microenvironment feature from each microenvironment image of the at least one microenvironment image;
utilize at least one prognosis inference machine learning model to infer a prognosis associated with the at least one microenvironment image using the at least one engineered microenvironment feature and the at least one derived compound microenvironment feature; and
generate a notification indicating the prognosis based on the at least one microenvironment image for display on a user computing device in communication with the at least one processor.
